(19) United States
(12) Patent Application Publication
(10) Pub. No.: US 2022/0023467 A1
(43) Pub. Date: Jan. 27, 2022

(54) ARTICULATED UV DISINFECTION DEVICE AND METHOD

(71) Applicant: Surfacide, LLC, Waukesha, WI (US)

(72) Inventors: Michael Scott Garner, Waukesha, WI (US); Waldemar John Lyslo, Waukesha, WI (US)

(73) Assignee: Surfacide, LLC, Waukesha, WI (US)

(21) Appl. No.: 17/382,249

(22) Filed: Jul. 21, 2021

Related U.S. Application Data
(60) Provisional application No. 63/054,685, filed on Jul. 21, 2020.

(51) Int. Cl. A61L 2/10

(52) U.S. Cl. CPC A61L 2/10; A61L 2202/14; A61L 2202/16; A61L 2202/11

(57) ABSTRACT

A device and method of cleaning a space and, in particular, successive rows of objects using light energy such as UV-C. The device includes an articulated boom that is adjustable along four degrees of freedom to optimize an orientation of the boom relative to the rows of objects.

### RELATED APPLICATIONS

This application claims benefit of and priority to U.S. Provisional Application Ser. No. 63/054,685 filed Jul. 21, 2020, entitled Articulated UV Disinfection Device, which is hereby incorporated herein by reference in its entirety.

### BACKGROUND OF THE INVENTION

The recent pandemic has brought to light the importance of maintaining clean surfaces. Certain environments, however, have a vast number of surfaces and cleaning them quickly is labor intensive. Examples of these environments, in addition to healthcare and long term care facilities, include stadiums, movie theaters, buses, subways, trains, aircraft, auditoriums, locker rooms, hotel rooms, tourist attractions, churches, classrooms, and numerous other environments with rows of seats, other objects and numerous fomites.

The use of UV-C (or this modality, e.g. UVGI-UV Germicidal Irradiation) has countless applications given the role that any surface plays in disease transmission. However for purposes of this application, we will use the minor example of an airplane to illustrate the problems being addressed by the invention, and understanding that the problems apply to many different environments including those listed above, cleanliness and the prevention of disease transmission is a major concern among all commercial airlines. A major concern of mass transit companies is keeping their vehicles clean and in service. For example, airlines want to maximize the amount of time the aircraft remain in the air. This requires a quick turnaround between landing and taking off on a subsequent flight. These two concerns conflict with each other because the cleaning procedure cannot take place until the aircraft has landed, the passengers and crew have debarked, and the maintenance crew in charge of cleaning the aircraft have boarded.

Conventional cleaning techniques used to clean aircraft between flights consist of picking up trash, and manual cleaning of surfaces with an EPA-approved disinfectant. When the aircraft is done flying at the end of the day, crews usually conduct a much more thorough cleaning as there are several hours before the plane takes off the next day.

Each environment has a window or opportunity and allocation of time to allow for manual cleaning, and to limit the continued exposure to further bioburden or environmental contamination. A room, car, bus, subway, airplane each requires a frequency of use and utilization which limits its downtime and availability for proper manual cleaning.

But now, in light of COVID-19, carriers are taking additional precautions to deeply clean and sanitize areas that passengers repeatedly touch or where they sit, stand, or even breathe. Many procedures are being published by health agencies such as the World Health Organization and the Centers for Disease Control. There are also ways to avoid germs once you're on board a plane that might help travelers avoid illness.

Some airlines, for example, have started spraying a high-grade disinfectant on every surface of the cabin after every flight. The disinfectant is sprayed through a fogging like machine or electro static sprayer that essentially aerosolizes the chemical so it can coat the air and all surfaces in the cabin, including the ceiling, seats, trays, floors, lavatories, crew rest areas, and galleys. Of course, care must be taken to ensure the fog adequately coats all of the surfaces. In addition, it is feared that the aerosolization of these chemicals will have long term health implications.

The recent pandemic has thus shown that conventional techniques are not adequately effective in disinfecting surfaces in order to prevent the transmission of infectious diseases. Repeatedly coating all of the cabin surfaces with chemicals may also not be free from unwanted consequences.

Introducing UV-C energy is an evidence-based way to manage the presence of bacteria, viruses and spores—including multi-drug resistant organisms. Disinfecting surfaces, such as those found in the cabin area of an airplane, can be performed by exposing the surfaces to UV-C energy—an evidence based modality—that is harmful to micro-organisms such as bacteria, viruses, fungi and spore. Ultraviolet germicidal irradiation (UVGI) is a proven disinfection method that uses ultraviolet (UV) energy at sufficiently short wavelengths to break-down and eradicate these organisms. It is believed that the short wavelength radiation disrupts cellular DNA or virus' RNA. It is also believed that UV energy works by destroying the nucleic acids in these organisms, thereby causing a disruption in the organisms' DNA. Once the DNA (or RNA) chain is disrupted, the organisms are unable to cause infection.

In addition to the effectiveness described above, there are advantages to using UV-C energy alone or in concert with other disinfection modalities. UV-C requires only electricity; there is no off-gassing of chemicals frequently associated with chemical based products. After a cabin is treated using UV-C energy, passengers may immediately board. Alternative disinfection modalities, on the other hand, often result in lingering chemicals or agents that must be cleared from the room prior to entry. UV-C energy leaves no residue, does not require drying time, cannot be spilled, requires little manpower to apply, requires very little skill on the part of the operator, and uses long-lasting bulbs that require very little inventory management.

Using UV-C energy to disinfect spaces with many objects, such as rows of seats, does present some unique problems. For example, two primary challenges impact efficacy and energy delivery of UV-C energy: shadows and distance. UV-C emitters may not be able to eradicate bacteria or viruses in shadowed areas because the energy is delivered along a line-of-sight. As such, shadowed areas must be reduced or eliminated for effective disinfection. In addition, the the UV-C device or appliance itself, from its own form/footprint and physical geometry, may itself create shadows. As such, one must consider address these shadows for effective delivery of UV-C energy.

Second, the attempt to introduce UV-C energy is dramatically impacted by the Inverse Square Law. This Law states that the intensity of the energy delivered to a surface is proportional to the inverse of the square of the distance between the energy source and the object. In other words, the energy received from the UV emitting source decreases exponentially as the distance is increased. Thus, if one object is twice as far away from a light source as another object, the further object receives only one quarter the energy as the closer object. Knowing specific energy levels are required to eradicate specific organism, this can dramatically impact efficacy.

As such, there is a need for surface UV disinfection system that exploits the advantages of UV energy, while also addressing the aforementioned problems.

More specifically, there is a need for an UV disinfection system that maximizes the effectiveness of the energy being emitted from its bulbs while eliminating shadows and reaching critical high touch passenger surfaces in a cabin despite fall-off due to distances from the light source(s).

### SUMMARY OF THE INVENTION

One aspect of the invention provides an articulated disinfection device for disinfecting an area comprising: a base assembly; an articulated light assembly connected to the base assembly with a swivel connection, the articulated light assembly including: a vertical mast; a boom having at least one disinfecting bulb; a connector connecting the boom to the mast; wherein the swivel connection allows the articulated light assembly to rotate around a vertical axis relative to the base assembly, thus providing a first degree of freedom; wherein the connector can swivel the boom relative to the mast thus providing a second degree of freedom; wherein the connector can slide up and down the vertical mast, thus providing a third degree of freedom; and, wherein the boom is connected to the connector with a hinge, thus providing a fourth degree of freedom.

In at least one embodiment, the base assembly comprises a control panel.

In at least one embodiment, the base assembly comprises a mobility component.

In at least one embodiment, the mobility component is motorized, thus allowing the base assembly to be self-propelled.

In at least one embodiment, the at least one bulb comprises at least one UV-C lamp.

In at least one embodiment, the at least one bulb is partially surrounded by a reflector.

In at least one embodiment, the control panel includes controls for controlling each of the four degrees of freedom.

In at least one embodiment, the reflector comprises a parabolic reflector.

In at least one embodiment, the device has a transport configuration in which the boom and mast are parallel, ensuring that a width of the device does not exceed a width of the base assembly.

In at least one embodiment, each of said swivel connection and said connector are motorized, allowing remote control of each of the four degrees of freedom.

Another aspect of the invention provides a device capable of disinfecting a plurality of rows of seats or fomites comprising: a self-propelled base assembly; an articulated light assembly connected to the base assembly and including: a vertical mast; a boom having at least one disinfecting bulb; a connector connecting the boom to the mast; wherein the boom has a plurality of degrees of freedom relative to the base assembly such that the boom can be adjustably positioned at a desired height and extending over a row of seats; wherein the self-propelled base assembly includes an adjustable speed control allowing the base assembly to propel itself down the aisle at a desired speed while the at least one disinfecting bulb sanitizes successive rows of seats.

In at least one embodiment, the self-propelled base assembly comprises a set of rear wheels and a set of front wheels.

One aspect of the invention is to provide automated propulsion or motorized movement. A set controlled speed provides a constant rate of exposure and energy delivery. Having adjustable speed control will affect the exposure rate as the emitter passes surfaces. This is control of exposure or dose by speed, and constant rate of output.

In at least one embodiment, the at least one set of wheels is powered.

In at least one embodiment, the front wheels are casters.

In at least one embodiment, the self-propelled base assembly comprises at least one scanner.

In at least one embodiment, the scanner is capable of detecting lateral limits of the aisle and providing steering instructions to the powered wheels.

Yet another aspect of the invention is a method of disinfecting successive rows of seats comprising: extending an articulated boom attached to a base assembly with at least four degrees of freedom and having at least one disinfecting bulb over a first row of seats at an optimal height above the first row of seats; energizing the disinfecting bulb; and propelling the base assembly along the aisle at a desired speed such that the boom passes over successive rows of seats.

In at least one embodiment, the desired speed is selected to ensure each row of seats receives an exposure amount sufficient to neutralize at least a predetermined pathogen.

In at least one embodiment, the base assembly is self-propelled and the step of propelling the base assembly is able to be accomplished by programming the desired speed into the base assembly.

In at least one embodiment, extending the articulated boom over the first row of seats comprises using controls to adjust each of the at least four degrees of freedom to optimize a position of the boom relative to the seats.

Any combination of the following desirable features have been incorporated into at least one of the embodiments of the invention. Nonlimiting examples include:

Untethered Operation—the device may be operated untethered from a power source;

On-Demand Operation—the device does not require being out of service for charging;

Low Time To Start Operating—the time needed for a user to start operating the device should allow for frequent use;

Ergonomic Movement—the device is able to be moved easily and ergonomically;

Moves Through Doors—the device is able to be moved through narrow cabin doors;

Moves Through narrow aisles such as Aircraft Aisles—the device is able to be moved through aisles;

Disinfects Floors—the device should be able to be operated so as to expose floor surfaces to UV-C light;

Disinfection Field—the device has a known/calculated area or field of disinfection;

Safety—the device shall be safe to operate or run;

Utilization Tracking—usage of the device shall have a record of lamp run time and other pertinent data shall be kept of its operation;

Cost Of Operation—the cost to operate the device shall be kept to a minimum;

Transport Logistics—the device must be able to be safely, economically, and efficiently transported to end-users from the manufacturer or warehouses;

Targeted Organisms—the device shall reduce the pathogenic bioburden of targeted organisms;

Validation of Energy Delivered—the device shall be able to measure the energy (directly or indirectly) delivered to the disinfection field;

Movement Over Floors—the device shall be able to be ergonomically moved over a variety of floors including deep pile carpet;

Can Be Moved Into ADA-Accessible Areas—the device shall be able to move to areas that are accessible to areas that are ADA compliant;

Elevator Thresholds—the device shall be able to move over the gaps common in elevator thresholds as well as thresholds seen in a public transportation;

Tracking Of Wear Components—the device includes wear components that can be tracked and displayed for the user;

Serviceable—the device can be serviced safely with minimal downtime, minimal cost, and where possible, without deployment of a technician;

Short Duration Of Use—the device is desired to minimize total time to completion of reduction of bioburden;

Movement On Inclines—the device shall be able to be safely moved over inclined surfaces;

Tethered Operation—the device shall be able to be run while charging or batteries are not present;

Minimal Self-Shadowing—the device shall have minimal shadowing of its own UV-C output;

Operational Climate Conditions—the device shall be able to be operated in environments with such extremes as Saudi Arabia's temperature and Mexico City's elevation;

Worldwide Operation—the device shall be able to be marketed and used in most countries;

Minimal Lamp Breakage—the device incorporates design elements to reduce and minimize accidental lamp breakage;

Modularity Supports Future Upgrades—the device shall be modular so as to have components upgraded or expanded in use for future development;

Detect Collisions—any automatic movements of the device shall detect collisions and stop or reverse that movement; If applicable, the user will be notified of the collision;

Push Mode—a user should be able to push the device to sweep the disinfection field through a path;

Multilingual—instructions and user interface shall be readable in multiple languages;

Elegant—the device incorporates Industrial design and aesthetics which are attractive and which embodies technological advancement;

Storage—the device must be reasonably and safely stored away when not in use;

Device Articulates—the device articulates;

Affordability—the device provides economic value and cost justified total cost of ownership which considers cost avoidance and an attractive return on investment;

Target Customers—some embodiments of the device are specifically designed for certain target customers for this device: airline services companies, hospitals, long-term care facilities, prisons, public transportation, skilled nursing care, athletics including wrestling mats and gymnastics surfaces, mosques, day care facilities, schools, playgrounds, martial arts studios, yoga studios, and the like;

Security—the device is secure for users to keep and use;

Electromechanical Articulation—the device has electromechanical articulation.

### BRIEF DESCRIPTION OF THE FIGURES

FIG. 1 is a perspective view of an embodiment of the present invention

FIG. 2 is side elevation of an embodiment of the present invention;

FIG. 3 is a close-up perspective view of an embodiment of the present invention;

FIG. 4 is a close-up perspective view of an embodiment of the present invention;

FIG. 5 is a plan view of an embodiment of a control panel of the invention;

FIG. 6 is a front elevation of a user disinfecting an aircraft using an embodiment of the invention;

FIG. 7. is a perspective view showing an example of an orientation of an embodiment of a boom of the invention;

FIG. 8. is a perspective view showing an example of an orientation of an embodiment of a boom of the invention;

FIG. 9. is a perspective view showing an example of an orientation of an embodiment of a boom of the invention;

FIG. 10. is a perspective view showing an example of an orientation of an embodiment of a boom of the invention;

FIG. 11. is a perspective view showing an example of an orientation of an embodiment of a boom of the invention; and,

FIG. 12 is a perspective view of an embodiment of the device showing access to a rechargeable battery.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

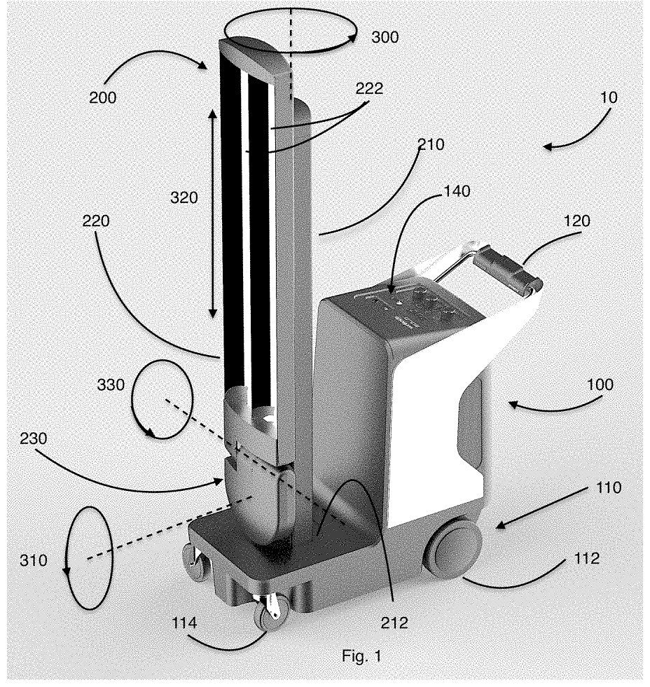

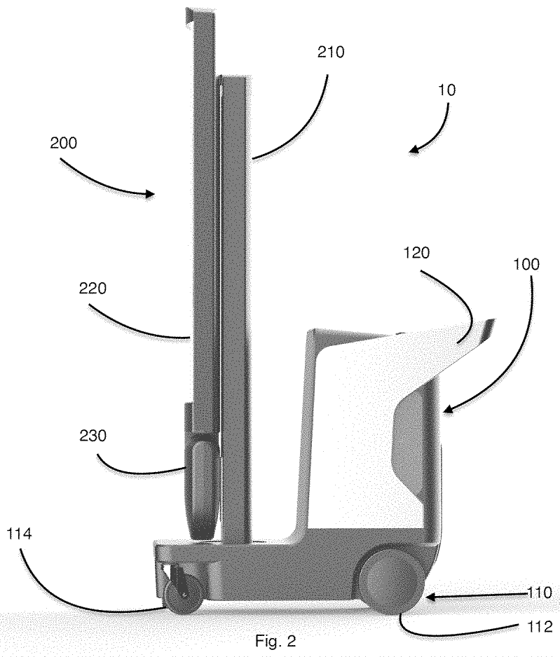

Referring now to FIGS. 1 and 2, there is shown an embodiment of a device 10 of the invention. Device 10 is a light tower that generally includes a base assembly 100 and a lamp assembly 200. In at least one embodiment, the base assembly includes a mobility component 110, such as wheels. The mobility component 110 shown in the figures includes two independent rear wheels 112 and two front swivel caster wheels 114. One skilled in the art will realize this wheel configuration is matter of design consideration and that other wheel configurations would not detract from the spirit of the invention.

In an alternate embodiment, wheels 112 and/or 114 are powered and directed by a drive unit (not shown) such as a motor. The motor is either controlled remotely by an operator or locally by an onboard navigation system. It is contemplated that the scanning system (discussed below) provides navigational input to the navigation system, allowing the device 100 to move around the room during the disinfection process in a computed manner calculated to eliminate shadow areas. Steering may be accomplished by driving the powered wheels at different speeds or in different directions, as is known in the art.

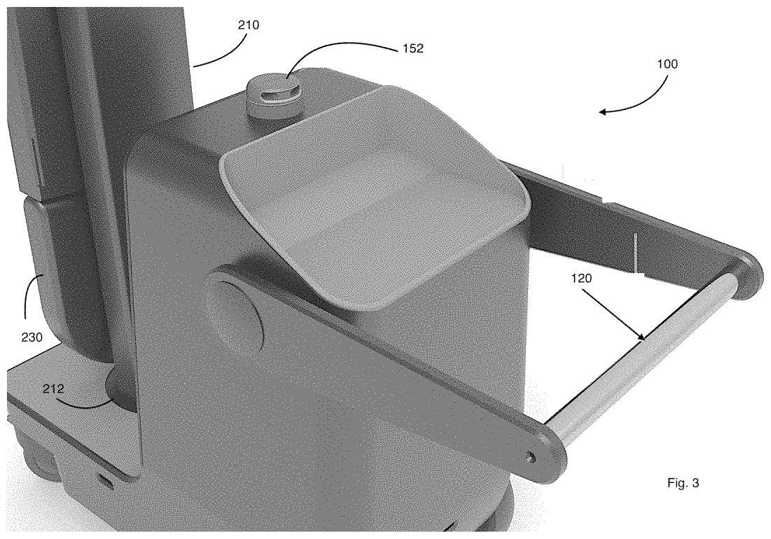

Also included is a handle 120. In some embodiments the handle is fixed relative to the base assembly 100, such as is shown in FIGS. 1 and 2. In other embodiments, such as shown in FIG. 3, the handle 120 is able to be pivoted or lowered relative to the base assembly 100, to accommodate different user heights and also to drop the handle out of the way if necessary to avoid the creation of shadows during use.

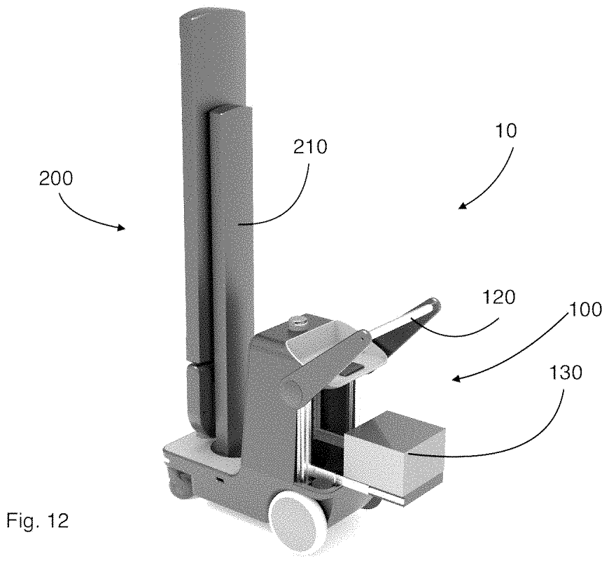

In at least one embodiment, the base unit further includes a power supply 130, such as a rechargeable battery (FIG. 12) or a cord usable to connect to an external power source (not shown). The rechargeable battery 130 is preferable for applications in which mobility is a priority. To allow rapid battery exchange, an access is provided, for example on a rear of the base assembly 100 as shown in FIG. 12. The corded embodiment may be preferable for applications involving long exposure times or short intervals between uses or to operate the device while charging the battery.

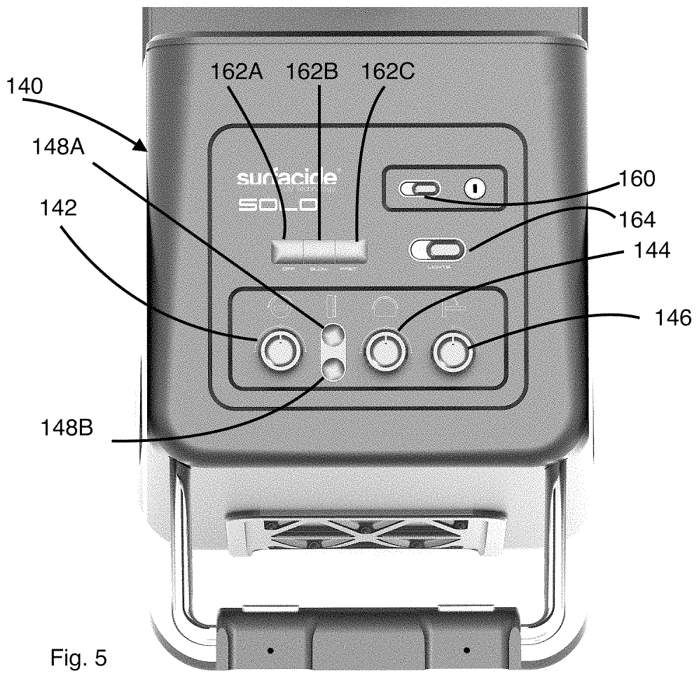

The base assembly may also include a control panel 140. As seen in FIG. 5, the control panel may include controls, shown as knobs and buttons 142, 144, 146, 148A, 148B, usable to control the various axes of freedom of the articulated light assembly 200, as explained further below. The control panel 140 may also include other controls for controlling the output of the light assembly 200. The output may be variable or fixed, programmed or manual, as explained in more details below. One skilled in the art will realize that any of wide variety of controls, instead of or in addition to knobs, may be used. Non-limiting examples include switches, buttons, sliders, toggles, etc.

For example, the embodiment of the control panel 140 shown in FIG. 5 further includes a power switch 160 and a set of movement control buttons 162A, 162B, and 162C. Button 162A is an “off” button used to stop movement. Button 162B is a “slow” button, causing the base assembly 100 to move slowly when depressed, and button 162C is a “fast” button, causing the base assembly 100 to move more quickly when depressed. Also shown is a “lights” switch 164.

In at least one embodiment, a speed adjustment, or time adjustment, will be provided either manually or automatically using a distance feedback loop tied to dose delivery. In other words, the dosage for a given surface will be calculated by an onboard processor, which will then adjust the speed.

In a preferred embodiment, the specific speeds of the base assembly 100 are programmed to target different pathogens. As different pathogens require varying levels of radiation to neutralize, the desired amount of radiation provided to a surface may be selected by varying the speed of the base assembly 100. A slower moving base assembly 100 will spend more time exposing a given surface to UV radiation. Alternatively or additionally, the amount of radiation provided may be varied by adjusting the intensity of the bulbs.

The base assembly 100 may further include safety features. For example, motion scanners, such as LIDAR, infrared, lasers, or other scanning technologies may be utilized to ensure that people are not exposed to disinfecting energy created by the device 10. For example, FIG. 3 shows a rear-facing scanner 152. The rear facing scanner scans behind the device for movement while the light assembly 200 is activated. If movement is detected, the light assembly 200 is immediately deactivated to prevent accidental exposure to the UV energy.

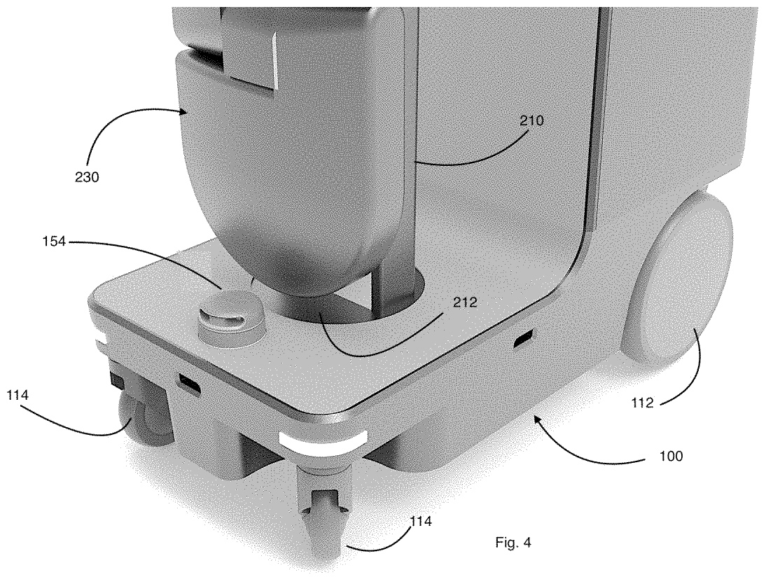

As seen in FIG. 4, the base assembly 100 may also include a forward-facing scanner 154. The forward-facing scanner 154 functions the same as the rear-facing scanner 152. If movement is detected ahead of the base assembly 100, the light assembly 200 is immediately deactivated to prevent accidental exposure to the UV energy.

In one embodiment, one or more of the scanners 152, 154, have the ability to control the direction of movement of the base assembly 100. For example, in self-propelled embodiments of the base assembly 100, the scanner 154 is able to detect aisle edges and keep the base assembly 100 in the middle of the aisle.

Referring to FIGS. 1 and 2, the articulated light assembly 200 generally includes a mast 210 that is connected to the base assembly 100 with a swivel connection 212. The swivel connection 212 is preferably motorized, the motor for which is hidden beneath the connection in the base assembly 100, or at the bottom of the mast assembly 210. The swivel connection 212 rotates along the axis of the mast 210 and thus provides a first degree of freedom 300 represented by a circular arrow.

The articulated light assembly 200 further includes a boom 220 with one or more bulbs 222 mounted thereto. The boom 220 is connected to the mast 210 with a connector 230 that can swivel relative to the mast 210 and slide up and down the mast 210 vertically. The connector 230 thus provides a second degree of freedom 310, represented by a circular arrow, and a third degree of freedom 320, indicated by a linear arrow.

The boom 220 is connected to the connector 230 with a hinge 232. The hinge 232 provides a fourth degree of freedom 330 represented by a circular arrow. The fourth degree of freedom 330 allows the boom to pivot away from the mast 210.

The bulb or bulbs 222, as seen in FIG. 1, emit UV-C light. Though the bulbs 222 shown utilize existing UV-C technology, one skilled in the art will realize that advancements in UV-C lamps could result in a variety of lamps being used with the invention. The embodiment of FIG. 1 uses two bulbs 222, each running parallel to a longitudinal axis of the boom 220. Behind each bulb 222 is a reflector 224. The reflectors 224 each wrap around a bulb 222 in order to focus and concentrate the light emitted from the bulbs 222 in a desired direction. Alternatively, a single reflector could be used that wraps around both bulbs 222. The reflector 224 may be parabolic, catenary, semi-circular, circular, or other curves, depending on the desired reflective result and/or the placement of the lamps. For example, a parabolic reflector, with the lamps located approximately close to the parabolic focal point, would result in a relatively narrow, focused (collimated) beam. Such a beam increases the intensity of UV radiation in a desired direction.

If desired, it is possible to incorporate a flatter reflector, such as a semi-sphere or catenary reflector. In this regard, a flexible reflector 224 may be provided that is connected to the boom 200 in a manner that allows the curve of the reflector to be adjusted based on the desired application.

Alternatively, beam adjustment or focusing could be accomplished by adjusting the lamp position relative to the reflector to create a “zoom” function that would allow the beam to be either more or less tightly focused.

In use, the device 10 is deployed by using the four degrees of freedom to optimally orient the boom relative to a surface to be cleaned and energizing the bulbs. Moving the cart during the cleaning process provides a fifth degree of freedom.

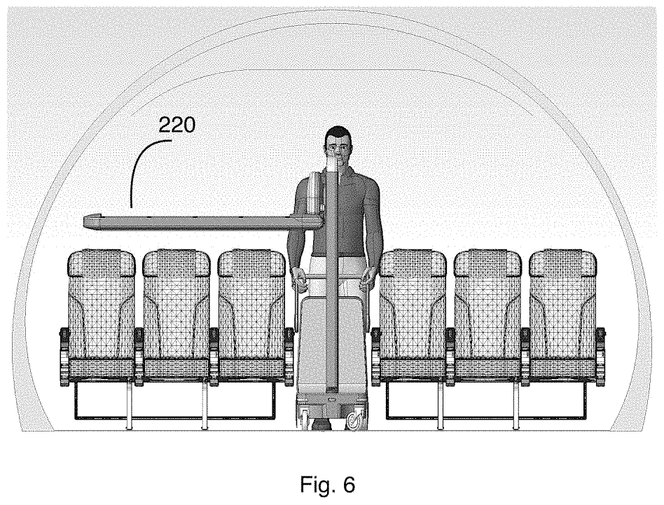

For example, as shown in FIG. 6, if successive rows of seats next to an aisle are to be cleaned, a method of disinfecting successive rows of seats may include extending the articulated boom over a first row of seats at an optimal height above the first row of seats. Then energizing the disinfecting bulb. Next the base assembly 100 is propelled along the aisle at a desired speed such that the boom 220 passes over successive rows of seats.

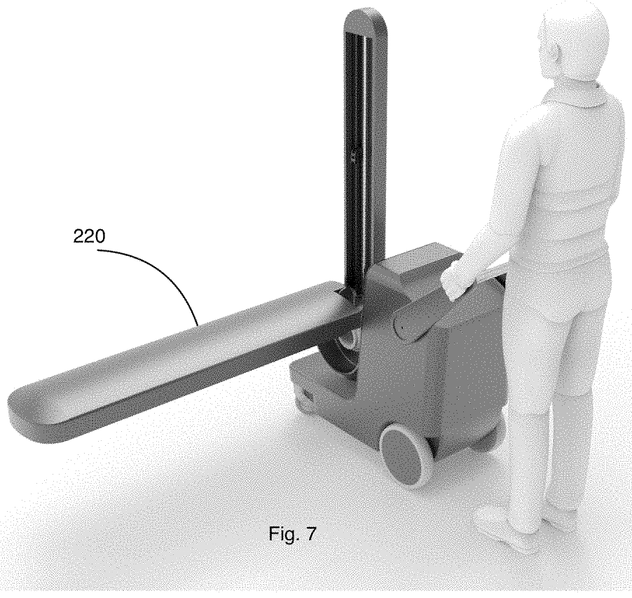

FIGS. 7-11 show the incredible flexibility provided by the four degrees of freedom of the lighting assembly 200 by illustrating various orientations of the boom 220 relative to the mast 210. FIG. 7 shows the boom 220 extended out to the side of the base assembly 100 and lowered to a height just above the floor. The boom 220 is facing downward such that the bulbs are facing the ground. This orientation is particularly useful for disinfecting a floor surface, such as at a daycare, mosque, or athletic surface.

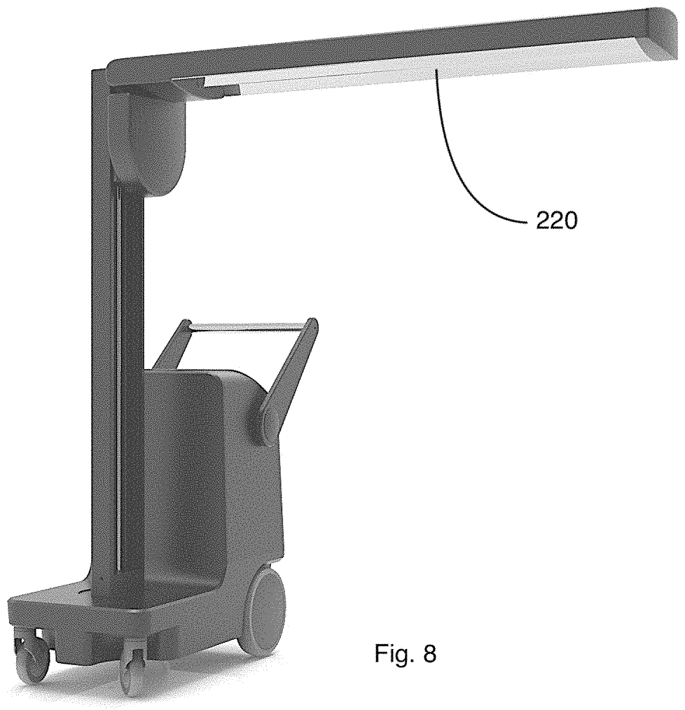

FIG. 8 shows the boom 220 extended out to the side of the base assembly 100 and raised to a maximum height above the floor. The boom 220 is facing downward such that the bulbs are facing the ground. This orientation is particularly useful for disinfecting an elevated surface such as a shelf or tier, or a plurality of tall objects.

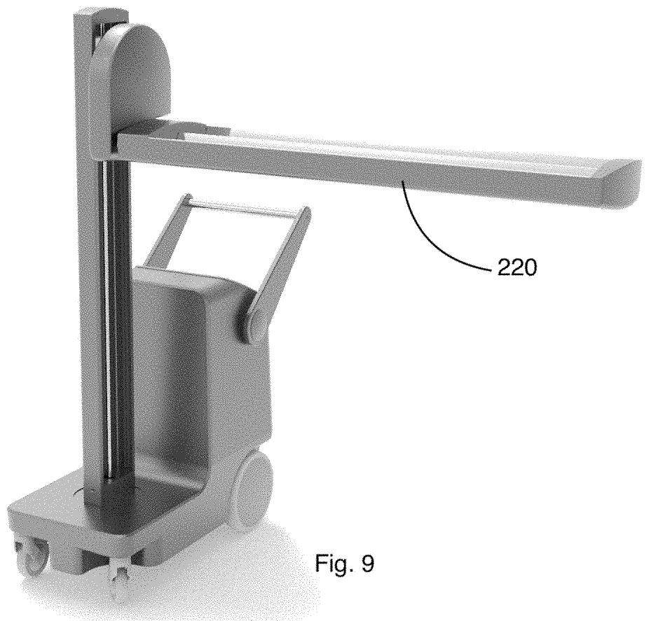

FIG. 9 shows the boom 220 extended out to the side of the base assembly 100 and raised to a maximum height above the floor. The boom 220 is facing upward such that the bulbs are facing the up. This orientation is particularly useful for disinfecting a low ceiling or overhead surface such as on an aircraft.

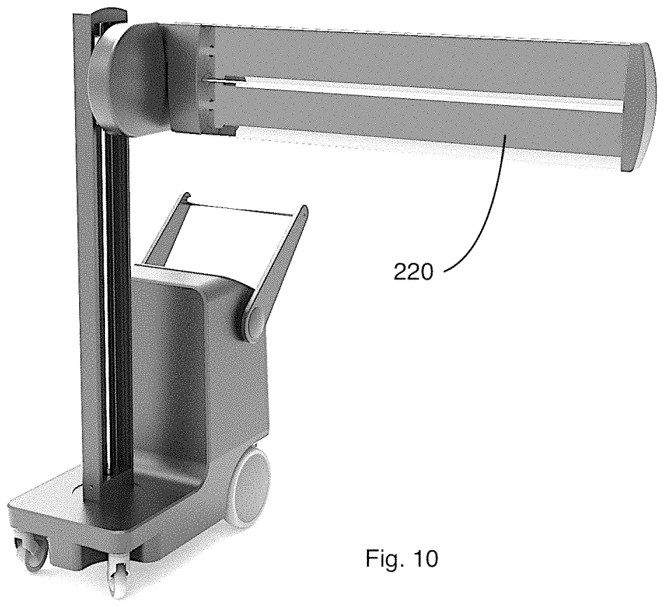

FIG. 10 shows the boom 220 extended out to the side of the base assembly 100 and raised to a maximum height above the floor. The boom 220 is facing upward such that the bulbs are facing the up. This orientation is particularly useful for disinfecting a vertical surface such as a wall.

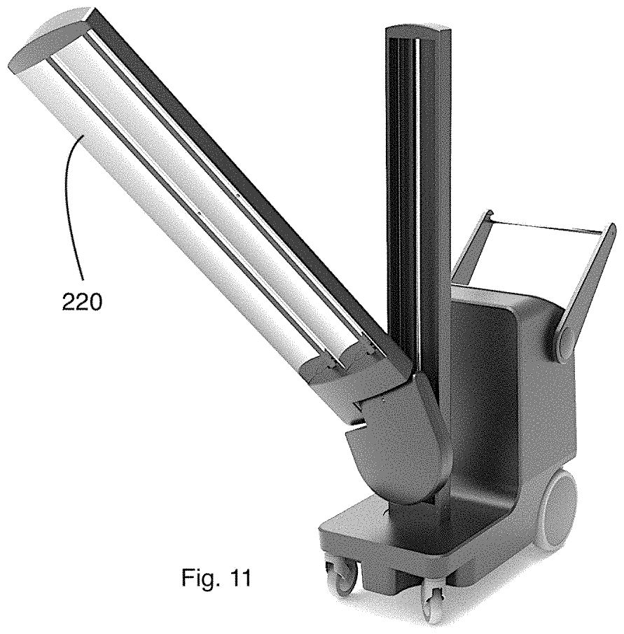

FIG. 11 shows the boom 220 at an oblique angle. This demonstrates the flexibility of the device and the infinite numbers of orientation provided by the various degrees of freedom.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An articulated disinfecting device for disinfecting an area comprising:
a base assembly;
an articulated light assembly connected to the base assembly with a swivel connection, the articulated light assembly including:
a vertical mast;
a boom having at least one disinfecting bulb;
a connector connecting the boom to the mast;
wherein the swivel connection allows the articulated light assembly to rotate around a vertical axis relative to the base assembly, thus providing a first degree of freedom;
wherein the connector can swivel the boom relative to the mast thus providing a second degree of freedom;
wherein the connector can slide up and down the vertical mast, thus providing a third degree of freedom; and,
wherein the boom is connected to the connector with a hinge, thus providing a fourth degree of freedom.

2. The device of claim 1 wherein said base assembly comprises a control panel.

3. The device of claim 1 wherein said base assembly comprises a mobility component.

4. The device of claim 3 wherein the mobility component is motorized, thus allowing the base assembly to be self-propelled.

5. The device of claim 1 wherein the at least one bulb comprises at least one UV-C lamp.

6. The device of claim 1 wherein the at least one bulb is partially surrounded by a reflector.

7. The device of claim 2 wherein the control panel includes controls for controlling each of the four degrees of freedom.

8. The device of claim 1 wherein said reflector comprises a parabolic reflector.

9. The device of claim 1 wherein said device has a transport configuration in which the boom and mast are parallel, ensuring that a width of the device does not exceed a width of the base assembly.

10. The device of claim 1 wherein each of said swivel connection and said connector are motorized, allowing remote control of each of the four degrees of freedom.

11. A device capable of disinfecting a space with a plurality of uneven surfaces comprising:
a self-propelled base assembly;
an articulated light assembly connected to the base assembly and including:
a vertical mast;
a boom having at least one disinfecting bulb;
a connector connecting the boom to the mast;
wherein the boom has a plurality of degrees of freedom relative to the base assembly such that the boom can be adjustably positioned at a desired height and extending over the uneven surfaces;
wherein the self-propelled base assembly includes an adjustable speed control allowing the base assembly to propel itself down an aisle or pathway among the uneven surfaces at a desired speed while the at least one disinfecting bulb sanitizes successive uneven surfaces.

12. The device of claim 11 wherein the self-propelled base assembly comprises a set of rear wheels and a set of front wheels.

13. The device of claim 12 wherein at least one set of wheels is powered.

14. The device of claim 12 wherein the front wheels are casters.

15. The device of claim 11 wherein said self-propelled base assembly comprises at least one scanner.

16. The device of claim 15 wherein said scanner is capable of detecting lateral limits of the aisle or pathway and providing steering instructions to the powered wheels.

17. A method of disinfecting successive rows of objects comprising:
extending an articulated boom attached to a base assembly with at least four degrees of freedom and having at least one disinfecting bulb over a first row of objects at an optimal height above the first row of objects;
energizing the disinfecting bulb;
propelling the base assembly along the aisle at a desired speed such that the boom passes over successive rows of objects.

18. The method of claim 17 wherein said desired speed is selected to ensure each row of objects receives an exposure amount sufficient to neutralize at least a predetermined pathogen.

19. The method of claim 17 wherein said base assembly is self-propelled and the step of propelling the base assembly can be accomplished by programming the desired speed into the base assembly.

20. The method of claim 17 wherein further comprising raising and lowering the articulated boom to maintain a desired distance over the objects being disinfected.
